(19) United States
(12) Patent Application Publication
(10) Pub. No.: US 2003/0186332 A1
(43) Pub. Date: Oct. 2, 2003

(54) Neurotrophic factor expression-inducing agent

(76) Inventors: Cristina Mitsumori, Chiba (JP); Noriyuki Morikawa, Ibarak (JP); Koji Hayashi, Osaka (JP); Kenji Nagahari, Tokyo (JP); Toshio Ota, Tokyo (JP); Yuri Hio, Hokkaido (JP); Tetsuo Nishikawa, Tokyo (JP); Takao Isogai, Ibaraki (JP); Masakazu Kawasaki, Kanagawa (JP); Kenji Hashimoto, Tokyo (JP); Toshimitsu Kishimoto, Tokyo (JP)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/239,487

(22) PCT Filed: Mar. 30, 2001
(86) PCT No.: PCT/JP01/02768

(30) Foreign Application Priority Data
Mar. 31, 2000 (JP) 2000-101300

(51) Int. Cl. G01N 33/53; G01N 33/567; A61K 38/18; A61K 48/00

(52) U.S. Cl. 435/7.2; 514/12; 514/44

(57) ABSTRACT

The culture supernatant of COS cells expressing the PSEC56 gene was found to have a neurite extension effect. The main substance of the activity contained in this culture supernatant was revealed to be NGF. It was shown that PSEC56 can be utilized as a neurotrophic factor expression-inducing agent, and that the NGF induction system can be utilized for drug development against diseases of the nervous system.

### TECHNICAL FIELD

The present invention relates to neurotrophic factor expression-inducing agents, a method for inducing the expression of neurotrophic factors, and a method of screening for compounds that regulate the induction of neurotrophic factor expression.

### BACKGROUND ART

Neurotrophic factor (NTF) is a factor that has the activity to maintain the survival and function of neurons, and promoting neurites extension. Currently, great attention has been paid on the factor as a target for drug development of nerve related diseases.

So far, neurotrophic factors, such as nerve growth factor (NGF), brain-derived neurotrophic factor (BDNF), NT-3, and NT-4/5, have been isolated. NGF, BDNF, NT-3, and NT-4/5 share a very similar physical and chemical structure with a similar physiological function, and are generically called neurotrophins. In addition, various cytokines, such as ciliary neurotrophic factor (CNTF), basic fibroblast growth factor (bFGF), epidermal growth factor (EGF), glial cell line-derived neurotrophic factor (GDNF), insulin-like growth factor-I and-II (IGF-I and -II, respectively) and interleukin-6 (IL-6) have been isolated.

Generally, administration of these isolated neurotrophic factors is widely tried to treat diseases accompanying neuronal degeneration and neuronal cell death, regardless of whether they belong to the central or peripheral nervous system. Furthermore, recently, although it is in the preclinical stages, gene therapy by neurotrophic factors is being planned. Moreover, use of neurotrophic factors for elevating the adhesion rate during transplantation of fetal neuron to the brain is also considered.

For example, the use of nerve growth factor (NGF) (Genentech/Hoffmann-La Roche) on diabetic peripheral neuropathy in clinical development reached phase III but did not proceed any further since no significant difference could be obtained. Currently, clinical application of NGF on HIV encephalopathy is proceeding. Further, no clinical effectiveness of brain-derived neurotrophic factor (BDNF) (Regeneron/Amgen, Sumitomo, Medtronic) on amyotrophic lateral sclerosis could be confirmed. However, further clinical tests are planned for BDNF on other general neuropathies, especially diabetic peripheral neuropathy. Regarding NT-3 (Regeneron/Amgen), clinical tests targeting diabetic peripheral neuropathy, amyotrophic lateral sclerosis, and such are in progress. Furthermore, clinical tests of ciliary neurotrophic factor (CNTF) (Cyto, Therapeutics) on amyotrophic lateral sclerosis and Huntington's disease are in progress.

On the other hand, drug development has been attempted using not only neurotrophic factors themselves, but also factors that induce the expression of a neurotrophic factor. At present, the production mechanism of neurotrophic factors and several compounds that promote the production of the factors are known. For example, a β2,3 agonist, clenbuterol, is known to promote NGF expression in glial cells via a β-adrenaline receptor. Increase of cyclic-adenosine monophosphate (cAMP) followed by the activation of cAMP-activated protein kinase (PKA) is reported to be involved in the pathway after adrenaline receptor stimulation. However, details of the pathway that ultimately leads to the expression of NGF are unknown. The activation of PKA via the β-adrenaline receptor is only an example of the pathways of NGF expression, and other molecular mechanisms involving various chemical substance and test systems that regulate the expression of neurotrophic factors, such as NGF, have been reported. Such mechanisms include pathways such as those involving PKA activation and cAMP increase by forskolin; those involving calcium-dependent protein kinase (PKC) activation by diacylglycerol and phorbol ester; and those via sphingomyelin involving vitamin D3, interleukin-1β (IL-1β), lipopolysaccharide, and ceramide (Semkova, I. and Krieglstein, J. (1999) Brain Res. Rev. 30, 176-188). In addition, aFGF is known to induce NGF production from glial cells (Neuroscience Letters 126, 18-20 (1991)). Furthermore, IL-1 and catecholamine are also known to induce the expression of NGF.

Xaliproden (SR-57746) is an example of neurotrophic factor expression-inducing agent that is being developed as a medicament. This substance is basically a 5-HT1A receptor agonist, and is reported to enhance NGF production via an unknown action mechanism to improve neuropathy in animal models. Clinical tests of Xaliproden for amyotropic lateral sclerosisandAlzheimer's disease are in progress. Application thereof to gene therapy by neurotrophic factors are also being considered.

As described above, drug development is being carried out regarding neurotrophic factors themselves and agents inducing them. Considering diseases of the central nervous system, however, neurotrophic factors are proteins of high molecular weight, and thus cannot pass through the blood brain barrier (BBB) in their original form. Therefore, when the neutrophic factors are administered to the periphery, they do not translocate into the brain and no effect against diseases of the central nervous system can be expected. Thus, the neurotrophic factors have to be injected directly into the brain for administration. On the other hand, inducing agents, if they are small molecules that go through the blood brain barrier, are expected to show effects on diseases of the central nervous system. Moreover, almost no effect can be anticipated by the administration of neurotrophic factors against diseases of the peripheral nervous system according to the results of clinical tests on peripheral neuropathy. Therefore, enhancing production of neurotrophic factors using inducing agents is more promising than the use of neurotrophic factors themselves.

Furthermore, continuous administration to the affected area is required for direct administration of neurotrophic factors. In contrast, constitutive expression of neurotrophic factors can be expected gene therapy and/or administration of inducing agents. Furthermore, if the inducing agents are small molecules, simplified (oral, subcutaneous, and such) administration enables maintenance of a necessary concentration in vivo, and as a consequence, constitutive expression of the neurotrophic factors can be expected.

Hitherto, regardless of their utility as medicaments, reports on neurotrophic factor expression-inducing agents are still few, and development of novel neuotrophic factor expression-inducing agents are desired.

### DISCLOSURE OF THE INVENTION

This present invention has been made in view of such situation, and its objective is to identify factors that induce the expression of neurotrophic factors to provide neurotrophic factor expression-inducing agents, method for inducing the expression of neurotrophic factors, and method of screening for compounds that regulate the induction of neurotrophic factor expression that utilize these identified factors.

The present inventors treated NT-2 nerve progenitor cells, human fetal testis-derived teratocarcinoma cells, with retinoic acid, prepared mRNAs from the treated cells, and then cloned a plurality of full-length cDNAs from the mRNAs by the oligo-capping method. The nucleotide sequences of these full-length cDNAs were analyzed, and a novel gene (PSEC56) with a secretory signal was discovered among them.

The inventors transfected the gene into COS cells, and examined the activity of the culture supernatant of COS cells expressing the gene on dorsal root ganglion (DRG) cells. As a result, this culture supernatant was revealed to have a neurite extension effect towards these cells. On the other hand, PSEC56 protein itself purified by metal affinity column from the culture supernatant did not show any neurite extension effect on DRG cells.

Accordingly, the present inventors performed analyses to identify the main substance of the activity included in this culture supernatant. As a result, the neurite extension effect of the culture supernatant on DRG cells was suppressed by treating the culture supernatant of PSEC56 transfected COS-7 cells with antibodies against NGF. Moreover, NGF was detected in the culture supernatant of COS-7 cells transfected with the PSEC56 gene. That is, the present inventors demonstrated NGF as the main substance of activity within the culture supernatant.

As described above, since NGF expression was induced by intracellular expression of PSEC56 protein, the PSEC56 protein and genes encoding said protein can be utilized as inducing agents of NGF expression in cells. Furthermore, the inducing system of NGF expression by PSEC56 protein expression may be also utilized for the development of therapeutic agents and preventive agents for diseases of the nervous system.

This invention is based on the above findings and specifically provides:

(1) a neurotrophic factor expression-inducing agent, wherein PSEC56 protein or DNA encoding said protein serves as an active ingredient;

(2) the agent of (1), wherein the neurotrophic factor is NGF;

(3) a method for inducing the expression of neurotrophic factors, which comprises the step of expressing PSEC56 protein;

(4) the method of (3), wherein the neurotrophic factor is NGF;

(5) a method of screening for compounds that regulate the induction of neurotrophic factor expression by PSEC56 protein, which comprises the steps of:

(a) contacting a test sample with a transfected cell including an expressive DNA encoding the PSEC56 protein;

(b) detecting the expression of the neurotrophic factor in said cell; and

(c) selecting the compound that enhances or suppresses the induction of neurotrophic factor expression compared to the expression detected in the absence of the test sample (control); and

(6) the method of (5), wherein the neurotrophic factor is NGF.

This invention provides neurotrophic factor expression-inducing agents, wherein PSEC56 protein or DNA encoding the protein serves as an active ingredient.

The active ingredient of an expression-inducing agent of this invention, “PSEC56 protein”, includes the PSEC56 protein of SEQ ID NO: 2, as well as proteins functionally equivalent thereto. Herein, “proteins functionally equivalent to PSEC56 protein of SEQ ID NO: 2” refers to proteins that are highly homologous, at the primary structure, to the PSEC56 protein of SEQ ID NO: 2 at the primary structure, and which, similarly to the PSEC56 protein of SEQ ID NO: 2, have the activity to induce neurotrophic factor expression upon their expression. It may be any protein with any kind of name. Herein, the term “highly homologous” refers to a sequence identity of at least 40% or more, preferably 60% or more and more preferably 80% or more (for example, 90% or more, or 95% or more). Examples of such proteins are mutants and variants of the PSEC56 protein of SEQ ID NO: 2, and homologous proteins derived from other organisms, but are not limited to these examples. The activity of a test protein to induce neurotrophic factors can be detected, for example, by expressing the test protein in COS cells, and detecting, as an indicator, secretion of neurotrophic factors, such as NGF, into the culture supernatant (see Example 2).

A protein that is functionally equivalent to the PSEC56 protein of SEQ ID NO: 2 can be prepared by one skilled in the art by, for example, a method for introducing mutations to the amino acid sequence of a protein (for example, site-directed mutagenesis (Current Protocols in Molecular Biology edit. Ausubel et al. (1987) Publish. John Wiley & Sons Section 8.1-8.5)). Such proteins may also be produced by naturally occurring amino acid mutations. The “PSEC56 protein” of this invention includes proteins that are functionally equivalent to the PSEC56 protein of SEQ ID NO: 2, in which one or more amino acids of the PSEC56 protein of SEQ ID NO: 2 have been substituted, deleted, inserted, and/or added. There are no limitations on the number of mutations and the sites of mutation of amino acids in the protein as long as its function is maintained. Typically, the number of mutations is 10% or less of all amino acids, preferably 5% or less of all amino acids, and more preferably 1% or less of all amino acids. Proteins with deletion of amino acids from the PSEC56 protein include, for example, proteins in which a signal peptide has been removed.

Additionally, a protein that is functionally equivalent to the PSEC56 protein of SEQ ID NO: 2 can be isolated using hybridization techniques or gene amplification techniques well known to those skilled in the art. That is, one skilled in the art can ordinarily isolate a DNA highly homologous to a probe from biological samples including human, rat, mouse, and such; and then, prepare the corresponding protein from the isolated DNA utilizing hybridization techniques wherein the DNA (SEQ ID NO: 1) encoding the PSEC56 protein of SEQ ID NO: 2 or parts thereof is used as a probe (Current Protocols in Molecular Biology edit. Ausubel et al. (1987) Publish. John Wiley & Sons Section 6.3-6.4).

A stringent hybridization condition for isolating DNAs encoding such proteins is usually “1×SSC, 0.1% SDS, 37° C.” or so, a more stringent condition “0.5×SSC, 0.1% SDS, 42° C.” or so, and an even more stringent condition “0.2×SSC, 0.1% SDS, 65° C.” or so. The more stringent the condition for hybridization, the more efficient isolation of a DNA with a higher homology with the probe sequence is expected. However, the above-mentioned combinations of SSC, SDS, and temperature conditions are only examples, and one skilled in the art can accomplish similar stringencies to those mentioned above by appropriately combining the above-mentioned and other factors (for example, probe concentration, probe length, hybridization reaction time, and so on) that determine the stringency of hybridization.

Alternatively, gene amplification techniques (PCR) (Current Protocols in Molecular Biology edit. Ausubel et al. (1987) Publish. John Wiley & Sons Section 6.1-6.4) may be used to amplify and isolate DNAs that are highly homologous to the DNA sequence (SEQ ID NO: 1) encoding the PSEC56 protein of SEQ ID NO: 2 or parts thereof by designing primers based on the information of the DNA sequence encoding the PSEC56 protein. Furthermore, using the isolated DNA, proteins that are functionally equivalent to the PSEC56 protein of SEQ ID NO: 2 can be obtained.

Proteins encoded by the DNAs that are isolated using such hybridization techniques and gene amplification techniques usually have high sequence homology at the amino acid sequence level to the PSEC56 protein of SEQ ID NO: 2.

A nucleotide sequence is determined as “homologous” by performing a sequence homology search with BLAST N, for example, when the sequence shows a homology of 40% or higher to a region of 400 bp or longer of the 1746 bp DNA region encoding the PSEC56 protein. On the other hand, an amino acid sequence can be determined “homologous” by performing a sequence homology search with BLAST X, for example, when the sequence indicates a homology of 30% or higher to a region of 80 residues or longer of the 582 residue of the PSEC56 protein. Furthermore, sequence homologies can be determined using other search algorithms, such as FASTA and Smith-Waterman (GenBank (http://www.ncbi.nlm.nih.gov/web/GenBank/)).

At the nucleotide sequence level, the PSEC56 sequence (see SEQ ID NOs: 1 and 2) shows sequence homologies of 49%, 48%, and 48% at 185 bp-751 bp, 552 bp-986 bp, and 1213 bp-1635 bp, respectively, to human FKBP12. The PSEC56 has a primary structure wherein three FKBP domains are aligned. In addition, on the C terminus of the amino acid sequence, a “His-Glu-Glu-Leu” sequence, which is predicted to be an endoplasmic reticulum translocation signal, has been confirmed. The PSEC56 sequence has 76% sequence homology (1-2632 bp), and 88% amino acid homology (1-582 residues) with mouse FKBP65.

As described above, the active ingredient of the expression-inducing agent of the present invention include proteins that are functionally equivalent to the PSEC56 protein of SEQ ID NO: 2, which proteins are encoded by DNAs that hybridize with a DNA encoding the PSEC56 protein of SEQ ID NO: 2.

The PSEC56 protein can be prepared as a recombinant protein, or as a naturally occurring protein. The recombinant protein can be prepared, for example, by introducing a vector to which a DNA encoding the PSEC56 protein has been inserted to an appropriate host cell, and then purifying the proteins expressed in the transfectant or those secreted into the culture supernatant. On the other hand, the naturally occurring protein can be prepared, for example, using an affinity column bound to antibodies against the PSEC56 protein (Current Protocols in Molecular Biology edit. Ausubel et al. (1987) Publish. John Wiley & Sons Section 16.1-16.19).

There are no particular limitations on the form of the “DNA encoding the PSEC56 protein”, which is an active ingredient of the expression-inducing agent of this invention, and includes cDNAs, as well as genomic DNAs, chemically synthesized DNAs, etc. The DNAs of this invention can be prepared by standard methods, such as hybridization methods using a DNA sequence (for example, SEQ ID NO: 1) encoding the PSEC56 protein or parts thereof as a probe, or PCR methods using primers synthesized based on the DNA sequence information.

There are no particular limitations on the form of the “DNA encoding the PSEC56 protein”, which is an active ingredient of the expression-inducing agent of this invention, so long as it is intracellularly expressible. Preferably, the DNA is inserted into a vector that ensures its intracellular expression. Preferably used vectors include pME18S FL3, pcDNA3.1(−), pcDNA3.1(+), etc.

The expression-inducing agent of this invention may be used as a reagent for experimental research. Alternatively, the agent can be used as a medicament for treatment or prevention of diseases, such as diseases of the nervous system. That is, the “expression-inducing agent” of this invention includes both reagents and medicaments.

Examples of neurotrophic factors to be induced by the expression-inducing agent of this invention are BDNF, NT-3, NT-4/5, CNTF, GDNF besides-NGF, but are not limited to these examples. The term “neurotrophic factors” herein refer to cell growth factors that act on cerebral nervous system and especially those having growth, differentiation, and (survival and/or functional) maintenance effects on neurons.

The present invention also provides a method to induce the expression of neurotrophic factors. This method includes the step of expressing the PSEC56 protein. There are no particular limitations on cells that are applicable for this method, and various cells are used according to the objective. For example, glial cells and Schwann cells that produce neurotrophic factors may be mainly used; but the present invention is not limited to these examples. To express the PSEC56 protein in a cell, for example, a DNA encoding the PSEC56 protein is inserted into a vector that ensures intracellular expression, and the vector is transfected into a target cell. Examples of conventional methods for the transfection of a vector into a cell includes the lipofectamine method, the calcium phosphate precipitation method, the electroporation method, the microinjection method, etc., but are not limited to these examples. For administration of a vector to patients, vectors derived from viruses such as retrovirus, adenovirus, and Sendai virus; and non-viral vectors such as liposomes may be used. In such cases, besides in vivo administration, ex vivo administration may also be conducted.

Additionally, the present invention provides a method of screening for compounds that regulate the induction of neutrophic factor expression caused by the PSEC56 protein. The screening method of this invention is characterized by the steps of adding a test sample to the above-mentioned expression induction system of neurotrophic factors, and then evaluating the effect of the test sample on the neurotrophic factor expression in response to PSEC56 expression. More specifically, the screening method of this invention can be carried out by the steps of: (a) contacting a test sample with a transfected cell that includes an expressive DNA encoding the PSEC56 protein; (b) detecting neurotrophic factor expression in the cell; and (c) selecting the compound that enhances or suppresses the induction of neurotrophic factor expression compared to the expression detected in the absence of the test sample.

Transfected cells used for the screening can be prepared by transfecting a cell with a vector to which a DNA encoding the PSEC56 protein has been inserted by the above-mentioned well-known methods for gene transfection. There are no particular limitations on the host cell to be transfected with a vector. Various cells are used according to the objective, and for example, COS cells and CHO cells are preferably used as eukaryotic cells for enhanced expression of the protein of interest. Alternatively, in vivo cells are exemplified by glial cells and Schwann cells that produce neurotrophic factors, but are not limited thereto.

There are no particular limitations on the test samples that are contacted with the cells prepared in this manner. For example, cell extracts, expression products of gene libraries, synthetic low molecular weight compounds, synthetic polypeptides, and naturally occurring compounds may be used, but is not limited to these examples.

According to the screening method of the present invention, the expression of a neurotrophic factor is detected after the contact of cells with a test sample. The expression of neurotrophic factors can be detected by performing Western Blotting using antibodies against the neurotrophic factors on the culture supernatant of transfected cells with the PSEC56 gene.

As a result of the detection, if the expression level of a neurotrophic factor is elevated compared to the level detected in the absence of the contact with a test sample (control), the used test sample is determined to promote the induction of the expression of the neurotrophic factor; and on the contrary, if the expression level of the neurotrophic factor is diminished compared to the control, the used test sample is determined to suppress the induction of the neurotrophic factor expression.

The PSEC56 protein and genes encoding the protein, as well as compounds isolated by the screening method of the present invention are expected to be applicable as therapeutic drugs and/or preventive drugs for spinal injury and diseases of the peripheral nerves, as well as diseases of the central nerve system including dementia, cerebral infarction, etc.

When using the PSEC56 gene for gene therapy, virus vectors such as retrovirus vector and adenovirus vector, or non-virus vectors such as liposomes, are used for administration to patients. Such administration methods include in vivo methods and ex vivo methods.

Alternatively, when using the PSEC56 protein and compounds isolated by the screening method of the present invention as medicaments, besides directly administering them to patients, they can be administered upon formulation by conventional preparation methods. For example, they can be administered orally or parenterally in forms such as tablets, capsules, granules, injections, and drops that are obtained via a normal preparation of medicinal components by mixing with pharmaceutical acceptable carriers (fillers, binders, disintegrators, corrigents, flavors, emulsifiers, and such), diluents, solubilizers, etc.

Tablets for oral administration include normally used carriers, such as sucrose, lactose, mannitol, maltitol, dextran, corn starch, and such; and typically include lubricants, such as magnesium stearate; preservatives, such as parabens and sorbic acids; antioxidants, such as ascorbic acid, α-tocopherol, and cysteine; disintegrators; binders; etc. Lactose and dried corn starch are effective diluents for capsule for oral administration.

Ordinary parenteral administrations, such as intravenous injections, intraperitoneal injections, and infusions, are prepared by appropriately adjusting the pH of the active ingredient solution, and then buffering and sterilizing the solution. Examples of vehicles and solvents that may be used include distilled water, Ringer solution, isotonic saline solution, etc. For use as intravenous injection, the total concentration of solute should be adjusted to make the solution isotonic.

The dose for administration is determined by taking the age, weight, administration time, method of administration, combination of drugs, and the condition of the disease of a patient to be treated, and other factors into consideration. The daily dose differs depending on the condition and weight of the patient, type of compound, administration route, and such, but for example, approximately 0.01 mg/patient/day to 1000 mg/patient/day, preferably 0.1 mg/patient/day to 300 mg/patient/day is preferred for oral administration; and approximately a dose of 0.01 mg/patient/day to 50 mg/patient/day, preferably 0.01 mg/patient/day to 10 mg/patient/day is preferably administered subcutaneously or intravenously for parenteral administration.

### BRIEF DESCRIPTION OF THE DRAWINGS

FIG. 1 shows the structure of the pME18SFL3 cloning vector. The stuffer was excised with DraIII and the cDNA was inserted into that region.

FIG. 2 shows the neurite extension effect of the culture supernatant of COS-7 cells transfected with PSEC56, measured by cultivating DRG cells in a media containing the culture supernatant.

FIG. 3 shows the neurite extension effect of the PSEC56 protein, measured by cultivating DRG cells in a media containing the protein.

FIG. 4 shows the neurite extension effect of the PSEC56 protein, measured by cultivating DRG cells in a media containing the protein.

FIG. 5 is a photograph confirming the existence of a fraction containing the Myc-His-PSEC56 protein by Western Blotting with anti-Myc antibodies.

### BEST MODE FOR CARRYING OUT THE INVENTION

Hereinafter, the present invention is specifically illustrated below with reference to Examples, but is not to be construed as being limited thereto.

### EXAMPLE 1

### Cloning of PSEC56

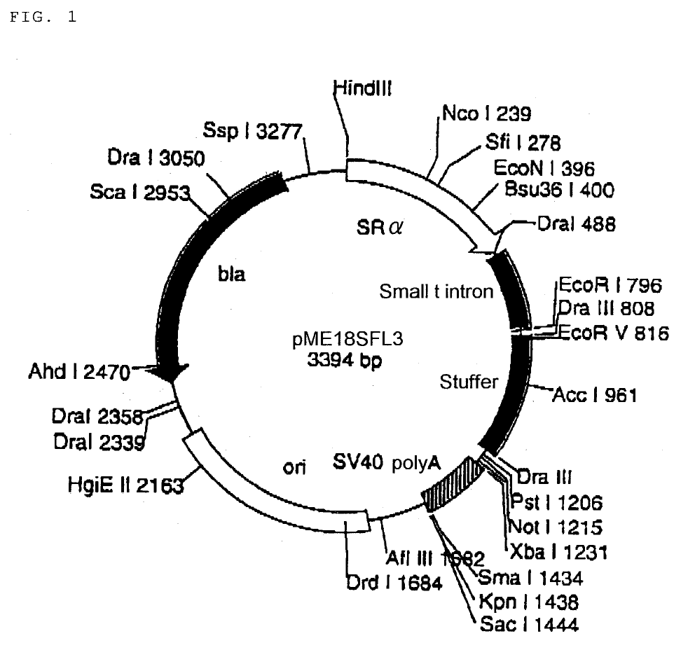

NT-2 nerve progenitor cells (purchased from Stratagene) which are human fetal testis-derived teratocarcinoma cells that can be differentiate into neurons by a treatment with retinoic acid, were used for the cloning of PSEC56. According to the attached instructions, NT-2 cells were cultivated, retinoic acid was added thereto, and the mixture was cultivated for another 2 weeks. These cultivated cells were collected to extract mRNAs according to the literature (J. Sambrook, E. F. Fritsch, and T. Maniatis, Molecular Cloning Second edition, Cold Spring Harbor Laboratory Press, 1989). Then, polyA(+) RNA was purified using oligo dT cellulose. cDNA was cloned from polyA(+)RNA by the oligo-capping method (M. Maruyama and S. Sugano, Gene, 138, 171-174 (1994)). Using oligo-cap linker (SEQ ID NO: 4) and oligo dT primer (SEQ ID NO: 5), BAP (Bacterial Alkaline Phosphatase) treatment, TAP (Tobacco Acid Phosphatase) treatment, RNA ligation, synthesis of primary strand cDNA, and removal of RNA were carried out as described in the literature (Suzuki and Sugano, Protein, Nucleic Acid, and Enzyme, 41, 197-201 (1996), Y. Suzuki et al., Gene, 200, 149-156 (1997)). Next, using 5′-(SEQ ID NO: 6) and 3′-(SEQ ID NO: 7) PCR primers, the primary strand cDNA was converted to double stranded cDNA by PCR (polymerase chain reaction), and then were digested with SfiI. Then, the cDNA was cloned, with a determined direction, into pME18SFL3 vector (GenBank AB009864, Expression vector, 3392 bp) digested with DraIII (FIG. 1). After a sequencing reaction of the obtained DNA using a DNA sequencing reagent (Dye Terminator Cycle Sequencing FS Ready Reaction Kit, PE Applied Biosystems) following the instructions, the nucleotide sequence of the DNA was analyzed using a DNA sequencer (ABI PRISM 377, PE Applied Biosystems). The longest possible nucleotide sequence from the 5′ end of each clone was determined by one pass sequencing. Then, cDNA clone PSEC56 was selected as a clone having a signal sequence by PSORT (Proc. Fourth Int. Conf. Intell. Sys. Mol. Biol., p. 109-115 (1996)) among clones indicating a score of 0.7 or higher according to the translation initiation ATG prediction software for cDNAs (ATGpr developed at Helix Research Institute (A. A. Salamov et al., Bioinformatics, 14, 384-390 (1998)) The full length nucleotide sequence of the cDNA clone was determined, and the amino acid sequence of the region encoding a protein (ORF) was predicted.

The nucleotide sequence of PSEC56, the amino acid sequence of the ORF predicted from the nucleotide sequence of PSEC56, and the predicted signal sequence of PSEC56 are shown in SEQ ID NOs: 1, 2, and 3, respectively.

### EXAMPLE 2

### Preparation of Cells

Preparation of dorsal root ganglion (DRG) cells was conducted according to the method of S. Kim (“No Shinkei Kenkyu no Purotocol: Baiyo kara Kino Kaiseki e (Protocols for Brain and Nerve Research, From Cell Cultivation to Functional Analysis)”, K. Mikoshiba, T. Shimizu, edition, Yodosha). More specifically, fetuses were extracted from a female rat (Crj: CD (SD) IGS) at day 18 of pregnancy, and DRGs were removed under a stereoscopic microscope. Cells were dispersed by treating the DRG with 0.25% trypsin. Upon removal of adherent cells other than neurocytes, the neurocytes were plated on a polylysine-coated 96-well plate (5,000 cells/well), and was cultivated for two days in D-MEM containing a test sample and 10% FCS in a CO₂ incubator. As a positive control, nerve growth factor (NGF) was used.

### EXAMPLE 3

### Measurement of the Neurite Extension Effect

Neurite extension of cells prepared by the above-mentioned method were measured by Cell-based ELISA using the expression of neurofilaments as an indicator of neurite extension. Specifically, cells were immobilized with formalin, were made highly permeable with a surfactant (Triton X-100), and were reacted with specific antibodies (SMI31; Sternberger) against neurofilaments. Next, the aforementioned antibodies were detected with peroxidase-labeled antibodies and peroxidase staining kit T (Sumitomo Bakelite) to measure the absorbance at 450 nm with a microplate reader.

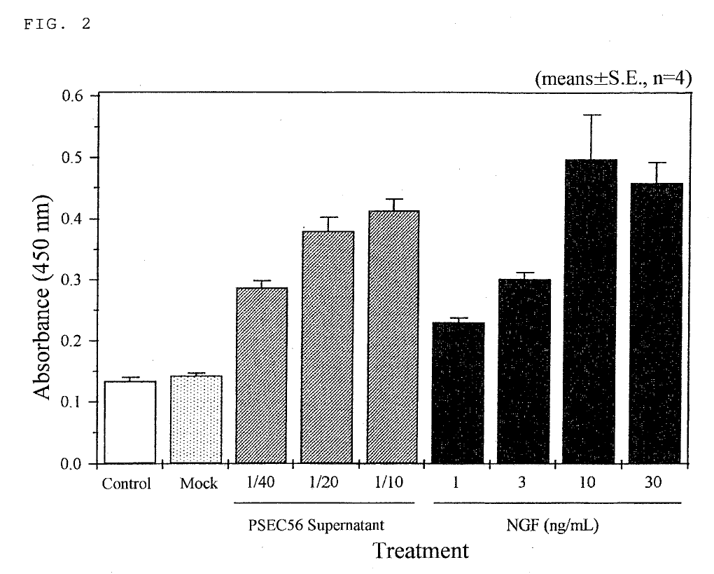

Similarly, to NGF, culture supernatant of COS-7 cells, which were transfected with the PSEC56 gene, as a test substance was revealed to have a neurite extension effect on DRG cells (FIG. 2).

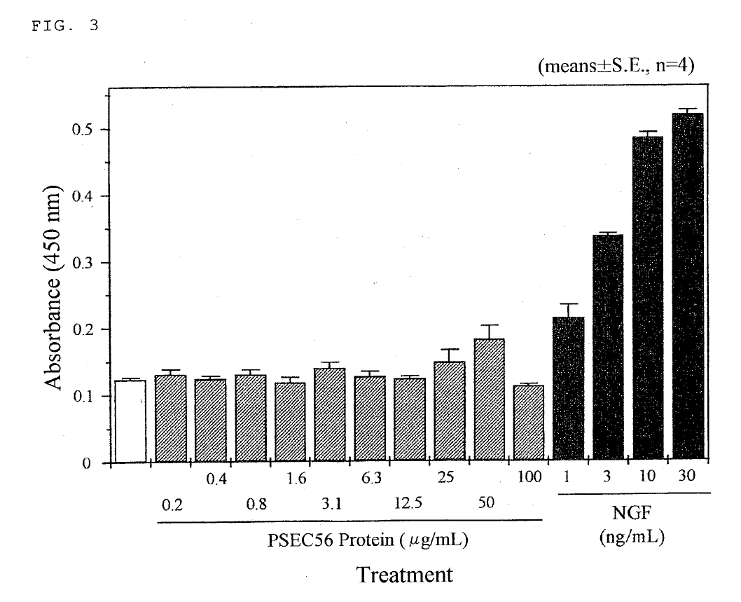

No neurite extension effect on DRG cells at all could be detected for the PSEC56 protein, which were purified from a culture supernatant with a metal affinity column, as a test substance (FIG. 3).

These results mentioned above indicate that not the PSEC56 protein itself has the effect to extend processes of neurocytes, but the main substance causing this effect exists as a molecule other than PSEC56 that is induced and synthesized by the transfection of the PSEC56 cDNA into cells.

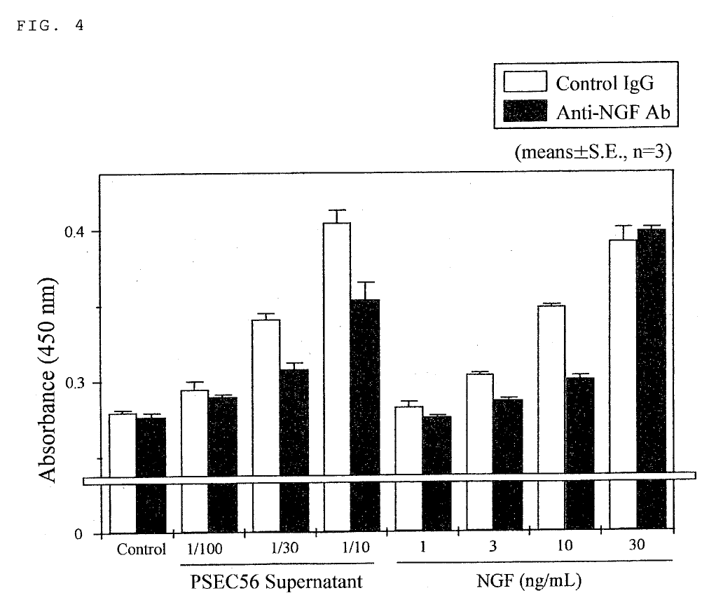

When a culture supernatant of PSEC56 gene-transfected COS-7 cells and the same culture supernatant treated with antibodies (Sigma) against NGF were used as the test substances, it was shown that the neurite extension effect on DRG cells was suppressed by the anti-NGF antibody. A similar effect was detected by other antibodies against NGF (FIG. 4).

### EXAMPLE 4

### Measurement of Nerve Growth Factors (NGF)

NGF in the culture supernatant were detected by Enzyme-Linked ImmunoSorbent Assay (ELISA) according to the instructions attached TM to NGF measurement kit from Promega (NGF Eₘₐₓ™ ImmunoAssay System) More specifically, a 96-well micro test plate was coated with anti-NGF polyclonal antibodies. After blocking, test substances were added thereto, and antigen-antibody reaction was carried out. Following the antigen-antibody reaction by the-addition of anti-NGF monoclonal antibodies, anti-rat IgG antibodies labeled with enzymes were added as secondary antibodies. Finally, a substrate, which forms colors through an enzyme reaction, was added thereto. NGF was quantified by measuring the absorbance of the color forming substrate.

When the culture supernatant of PSEC56 gene-transfected COS-7 cells was used as a test substance, NGF was detected from the culture supernatant (approximately 100 ng/mL). However, no NGF could be detected by the Mock transfection.

The results of this Example and FIG. 4 showed that the main molecule having the effect of neurite extension is NGF.

### EXAMPLE 5

### Purification of PSEC56 Protein

The DNA of PSEC56 was inserted into a vector for the addition of Myc and polyhistidine tag (pcDNA3.1(−)/MycHis-A; INVITROGEN). The expression vector was introduced into COS-7 cells by the calcium phosphate method or by the lipofectamine method. 3 days later, the culture supernatant was collected.

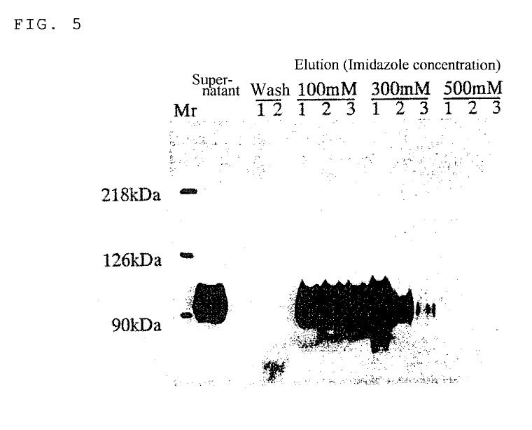

The polyhistidine-attached PSEC56 protein was purified from this culture supernatant using a metal affinity column (HisTrap; Amersham Pharmacia). Specifically, the culture supernatant was passed through a HisTrap column to which nickel ion was added, and the Myc-His-PSEC56 protein bound to the column was eluted using a desired concentration of imidazole solution. Fractions containing the Myc-His-PSEC56 protein were confirmed by Western Blotting using SDS-PAGE and anti-Myc antibody (FIG. 5). Since imidazole was used for the elution from the column, after collecting the fractions of interest, they were desalted by gel filtration using a PD-10 column (Amersham Pharmacia) for later experiments.

### INDUSTRIAL APPLICABILITY

The present invention provides neurotrophic factor expression-inducing agents, a method for inducing the expression of neurotrophic factors, and a method of screening for compounds that regulate the induction of neurotrophic factor expression utilizing PSEC56. This invention enables elucidation of the mechanism leading to the induction and secretion of neurotrophic factors, such as NGF, by PSEC56; and development of novel therapeutic drugs and preventive drugs for diseases of the central nervous system, such as dementia and cerebral infarction, as well as spinal injury and diseases of the peripheral nerves.

1. A neurotrophic factor expression-inducing agent, wherein PSEC56 protein or DNA encoding said protein serves as an active ingredient.

2. The agent of claim 1, wherein the neurotrophic factor is NGF.

3. A method for inducing the expression of neurotrophic factors, which comprises the step of expressing PSEC56 protein.

4. The method of claim 3, wherein the neurotrophic factor is NGF.

5. A method of screening for compounds that regulate the induction of neurotrophic factor expression by PSEC56 protein, which comprises the steps of:
(a) contacting a test sample with a transfected cell including an expressive DNA encoding the PSEC56 protein;
(b) detecting the expression of the neurotrophic factor in said cell; and
(c) selecting the compound that enhances or suppresses the induction of neurotrophic factor expression compared to the expression detected in the absence of the test sample (control).

6. The method of claim 5, wherein the neurotrophic factor is NGF.


SEQUENCE LISTING (7 sequences)

SEQ ID NO: 1 - DNA, 2641 bp, Homo sapiens
  CDS (87)...(1832)
gagccagttctgggaggcggggggaaggaggttggtggcgactccctcgctcgccctcactgccggcggtcccaactccaggcaccatgttccccgcgggcccccccagccacagcctcctccggctccccctgctgcagttgctgctactggtggtgcaggccgtggggagggggctgggccgcgccagcccggccgggggccccctggaagatgtggtcatcgagaggtaccacatccccagggcctgtccccgggaagtgcagatgggggattttgtgcgctaccactacaacggcacttttgaagatggcaagaagtttgattcaagctatgatcgcaacaccttggtggccatcgtggtgggtgtggggcgcctcatcactggcatggaccgaggcctcatgggcatgtgtgtcaacgagcggcgacgcctcattgtgcctccccacctgggctatgggagcatcggcctggcggggctcattccaccggatgccaccctctacttcgatgtggttctgctggatgtgtggaacaaggaagacaccgtgcaggtgagcacattgctgcgcccgccccactgcccccgcatggtccaggacggcgactttgtccgctaccactacaatggcaccctgctggacggcaccttcttcgacaccagctacagtaagggcggcacttatgacacctacgtcggctctggttggctgatcaagggcatggaccaggggctgctgggcatgtgtcctggagagagaaggaagattatcatccctccattcctggcctatggcgagaaaggctatgggacagtgatccccccacaggcctcgctggtctttcacgtcctcctgattgacgtgcacaacccgaaggacgctgtccagctagagacgctggagctcccccccggctgtgtccgcagagccggggccggggacttcatgcgctaccactacaatggctccttgatggacggcaccctcttcgattccagctactcccgcaaccacacctacaatacctatatcgggcagggttacatcatccccgggatggaccaggggctgcagggtgcctgcatgggggaacgccggagaattaccatccccccgcacctcgcctatggggagaatggaactggagacaagatccctggctctgccgtgctaatcttcaacgtccatgtcattgacttccacaaccctgcggatgtggtggaaatcaggacactgtcccggccatctgagacctgcaatgagaccaccaagcttggggactttgttcgataccattacaactgttctttgctggacggcacccagctgttcacctcgcatgactacggggccccccaggaggcgactctcggggccaacaaggtgatcgaaggcctggacacgggcctgcagggcatgtgtgtgggagagaggcggcagctcatcgtgcccccgcacctggcccacggggagagtggagcccggggagtcccaggcagtgctgtgctgctgtttgaggtggagctggtgtcccgggaggatgggctgcccacaggctacctgtttgtgtggcacaaggaccctcctgccaacctgtttgaagacatggacctcaacaaggatggcgaggtccctccggaggagttctccaccttcatcaaggctcaagtgagtgagggcaaaggacgcctcatgcctgggcaggaccctgagaaaaccataggagacatgttccagaaccaggaccgcaaccaggacggcaagatcacagtcgacgagctcaagctgaagtcagatgaggacgaggagcgggtccacgaggagctctgaggggcagggagcctggccaggcctgagacacagaggcccactgcgagggggacagtggcggtgggactgacctgctgacagtcaccctccctctgctgggatgaggtccaggagccaactaaaacaatggcagaggagacatctctggtgttcccaccaccctagatgaaaatccacagcacagacctctaccgtgtttctcttccatccctaaaccacttccttaaaatgtttggatttgcaaagccaatttggggcctgtggagcctggggttggatagggccatggctggtcccccaccatacctcccctccacatcactgacacagctgagcttgttatccatctccccaaactttctctttctttgtacttcttgtcatccccactcccagcccctattcctctatgtgacagctggctaggacccctctgccttcctccccaatcctgactggctcctagggaaggggaaggctcctggagggcagccctacctctcccatgccctttgccctcctccctcgcctccagtggaggctgagctgaccctgggctgctggaggccagactgggctgtagttagcttttcatccctaaagaaggctttccctaaggaaccatagaagagaggaagaaaacaaagggcatgtgtgagggaagctgcatgggtgggtgttagggctatgaaatcttggatttggggctgaggggtgggagggagggcagagctctgcacactcaaaggctaaactggtgtcagtccttttttcctttgttccaaataaaagattaaaccaatggc

SEQ ID NO: 2 - protein, 582 aa, Homo sapiens
MFPAGPPSHSLLRLPLLQLLLLVVQAVGRGLGRASPAGGPLEDVVIERYHIPRACPREVQMGDFVRYHYNGTFEDGKKFDSSYDRNTLVAIVVGVGRLITGMDRGLMGMCVNERRRLIVPPHLGYGSIGLAGLIPPDATLYFDVVLLDVWNKEDTVQVSTLLRPPHCPRMVQDGDFVRYHYNGTLLDGTFFDTSYSKGGTYDTYVGSGWLIKGMDQGLLGMCPGERRKIIIPPFLAYGEKGYGTVIPPQASLVFHVLLIDVHNPKDAVQLETLELPPGCVRRAGAGDFMRYHYNGSLMDGTLFDSSYSRNHTYNTYIGQGYIIPGMDQGLQGACMGERRRITIPPHLAYGENGTGDKIPGSAVLIFNVHVIDFHNPADVVEIRTLSRPSETCNETTKLGDFVRYHYNCSLLDGTQLFTSHDYGAPQEATLGANKVIEGLDTGLQGMCVGERRQLIVPPHLAHGESGARGVPGSAVLLFEVELVSREDGLPTGYLFVWHKDPPANLFEDMDLNKDGEVPPEEFSTFIKAQVSEGKGRLMPGQDPEKTIGDMFQNQDRNQDGKITVDELKLKSDEDEERVHEEL

SEQ ID NO: 3 - protein, 26 aa, Homo sapiens
MFPAGPPSHSLLRLPLLQLLLLVVQA

SEQ ID NO: 4 - RNA, 30 nt, Artificial Sequence
  an artificially synthesized oligo-cap linker sequence
agcaucgagucggccuuguuggccuacugg

SEQ ID NO: 5 - DNA, 42 bp, Artificial Sequence
  an artificially synthesized oligo(dT) adapter primer sequence
gcggctgaagacggcctatgtggccttttttttttttttttt

SEQ ID NO: 6 - DNA, 21 bp, Artificial Sequence
  an artificially synthesized primer sequence used to clone PSEC56
agcatcgagtcggccttgttg

SEQ ID NO: 7 - DNA, 21 bp, Artificial Sequence
  an artificially synthesized primer sequence used to clone PSEC56
gcggctgaagacggcctatgt